(19) United States
(12) Patent Application Publication
(10) Pub. No.: US 2006/0264971 A1
(43) Pub. Date: Nov. 23, 2006

(54) Intraocular lens injection nozzle

(76) Inventor: Takayuki Akahoshi, Tokyo (JP)

Correspondence Address: Jerry A. Schulman;Terrace Executive Center, Court C, One South 376 Summit Avenue, Oakbrook Terrace, IL, 60181, US

(21) Appl. No.: 11/320,106

(22) Filed: Dec. 28, 2005

Related U.S. Application Data
(60) Provisional application No. 60/682,179, filed on May 18, 2005.

(51) Int. Cl. A61F 9/00

(52) U.S. Cl. 606/107

(57) ABSTRACT

An improved nozzle for injecting an IOL through a corneal incision and into a human eye has a nozzle with upper, or upper and lower tapers formed at the end thereof to facilitate passage of the nozzle into the incision. A textured surface formed on the upper taper helps to keep the nozzle from slipping when it is inserted.

This invention relates to foldable intraocular lenses and, more particularly, to improved nozzles for injectors and cartridges used for injecting intraocular lenses into the eye. Applicant claims priority from U.S. Provisional Patent Application entitled “Improved Intraocular Lens Injection Nozzle”, Ser. No. 60/682,179, filed May 18, 2005.

### BACKGROUND OF THE INVENTION

A common ophthalmological surgical technique for treating cataracts and other diseases of the lens is to fractionate, emulsify and remove the diseased lens and replace it with a synthetic, plastic lens. To do so, an incision is made in the eye through which the diseased lens is removed and through which the new lens is inserted.

It is desirable to make the incision for removing the diseased lens and inserting the new lens as small as possible. This aids in shortening recovery time and limits the leakage of fluid through the incision after surgery without requiring sutures.

When originally introduced, replacement intraocular lenses (IOLs) were made from rigid plastics such as polymethylmthacrylate and required a relatively large corneal incision through which the lens would be inserted. Such incisions had to be sutured to limit the loss of fluid from the eye. The development of the soft lens, made from materials such as hydrogels, silicones and soft acrylics made it possible to fold or roll the lens prior to insertion. It is now a common surgical technique to fold such an IOL, insert it through the incision and allow the lens to unfold and position itself within the eye. When folded, the lens needs a much smaller incision for insertion than if the lens were inserted in its fully unfolded state.

Examples of foldable IOLs and systems for injecting them are well represented in the prior art.

U.S. Pat. No. 5,947,976 (Van Noy et al) teaches and describes an asymmetric IOL injection cartridge having an asymmetric bore. The IOL is inserted into the cartridge in its unfolded state, and when pushed through the cartridge, is partially folded when it exits the cartridge tip.

U.S. Pat. No. 5,976,150 (Copeland) teaches and describes an IOL injection system using a foldable substrate to compress and fold an IOL around an outer edge of the substrate. The folded assembly is then placed in an injector and expressed through the injector nozzle into the eye.

U.S. Pat. No. 6,083,231 (Van Noy et al) teaches and describes an asymmetric IOL injection cartridge. This patent is a continuation in part of the previously described U.S. Pat. No. 5,947,976 and adds to the disclosure in the '976 patent a peg used to engage the haptic on an IOL to keep the haptic in position as the lens is injected through the injector nozzle.

U.S. Pat. No. 6,143,001 (Brown et al) teaches and describes an asymmetric intraocular lens injection cartridge. This patent is a continuation in part of the previously described U.S. Pat. No. 6,083,231 as well as the '976 patent. Brown et al adds to the teachings of the prior mentioned patents a modified asymmetric bore which acts to fold the IOL as it is forced through the bore and the cartridge nozzle.

U.S. Pat. No. 6,398,789 (Capetan) teaches and describes an IOL injector cartridge similar in construction to the cartridge shown in the '976, '231 and '001 patents and which adds to the elements of those patents a heat-retention agent to keep the IOL warmed during the injection process.

U.S. Pat. No. 6,537,283 (Van Noy) teaches and describes an IOL shipping case and injection cartridge comprising an injection cartridge to which a case for an unfolded IOL is hingedly attached. The case can be rotated to align the lens with the central bore of the cartridge to let the lens thereafter be pushed through the cartridge and expressed through the cartridge tip.

U.S. Pat. No. 4,681,102 (Bartell) teaches and describes apparatus and method for insertion of an IOL where the system consists of an injector and a foldable load chamber within which the unfolded lens is placed. The chamber is then folded shut thereby folding the lens in half and a plunger is used to force the lens from the load chamber through a tip and into the eye.

These IOL delivery systems share a common characteristic: however the IOL is manipulated through the injector or cartridge, it inevitably is expressed or discharged through a nozzle into the eye. This is accomplished by positioning the tip of the nozzle at the incision, passing the nozzle into or through the incision into the anterior chamber and then expressing the IOL from the cartridge and through the nozzle allowing the IOL to unfold and position itself properly.

Although such fold-and-deliver systems have made it possible to use a smaller incision, the incision must still be of a size to allow the nozzle tip to pass therethrough. The present invention improves this process by shaping the nozzle tip to more easily be accommodated by the incision and to make possible the use of even smaller incisions.

### SUMMARY OF THE INVENTION

The present invention comprises a nozzle having a tip which may be used as the exit nozzle from an IOL-holding cartridge or from an injector through which an already folded IOL is forced. The tip features an exterior taper which minimizes the size of the nozzle at the incision allowing the tip to be more easily inserted through the incision. Thus, the incision need not be large enough to accommodate the major dimension of the nozzle that can be stretched as the tip is inserted through the incision and thereafter returned to its original size when the nozzle tip is withdrawn from the incision. A textured surface is formed on the upper taper to inhibit the nozzle from slipping when it is inserted into the incision.

These and other advantages of the present invention will become apparent upon consideration of the accompanying descriptions and the accompanying drawings in which:

### BRIEF DESCRIPTION OF THE DRAWINGS

FIG. 1 is a side elevational view of a prior art IOL injection cartridge;

FIG. 2 is a top plan view of the prior art cartridge shown in FIG. 1;

FIG. 3 is a sectional view taken along line 3-3 of FIG. 2;

FIG. 4 is a partial sectional schematic view illustrating the placement of the nozzle tip at the incision;

FIG. 5 is a partial sectional schematic view demonstrating a first preferred embodiment of the present invention having a partial upper exterior taper;

FIG. 5A is an end view of the nozzle of FIG. 5;

FIG. 6 is a partial sectional schematic of a second preferred embodiment of the present invention, having a partial lower exterior taper;

FIG. 6A is an end view of the nozzle of FIG. 6

FIG. 7 is a partial sectional schematic view of a third preferred embodiment of the present invention showing both upper and lower exterior tapers;

FIG. 7A is an end view of the nozzle of FIG. 7;

FIG. 8 is a partial sectional schematic of a fourth preferred embodiment of the present invention, having a roughened, textured surface formed on the upper exterior taper;

FIG. 9 is an end view of a nozzle such as shown in FIG. 8 with a textured surface formed by upstanding bumps; and

FIG. 10 is a partial sectional schematic showing the insertion of the nozzle of FIG. 8 into an eye.

### DETAILED DESCRIPTION OF THE INVENTION

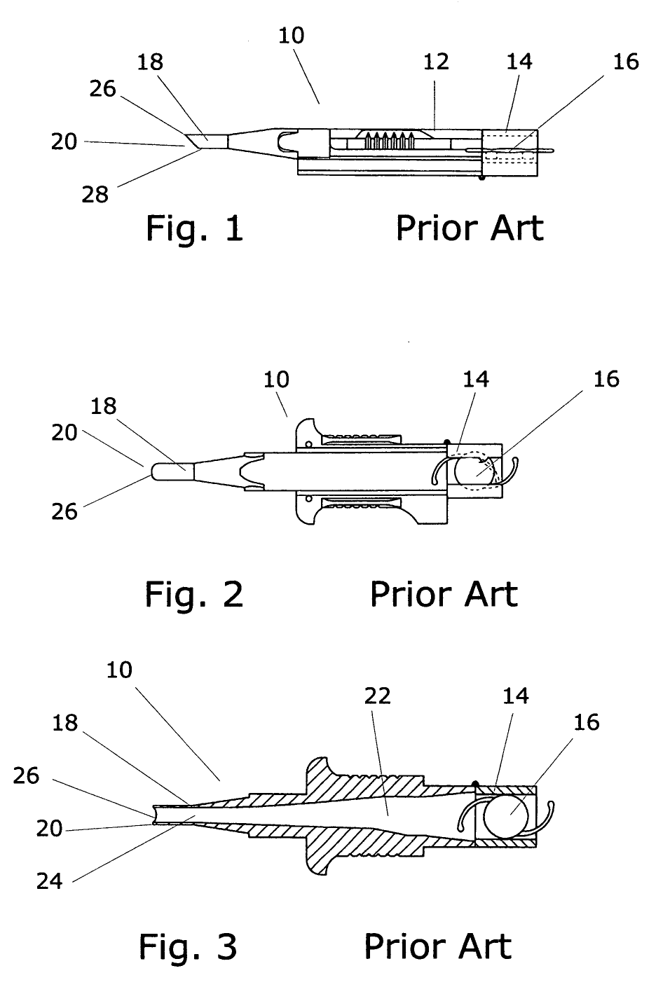

Referring now to FIG. 1, the numeral 10 identifies generally a prior art intraocular lens injection cartridge as shown and described in U.S. Pat. No. 6,537,283. Cartridge 10 has a hollow body 12 to which is attached a lens holder 14 and within which an IOL 16 is held. As seen in FIGS. 2 and 3, this particular injection system allows the cartridge 10 to accept IOL 16 in its unfolded state and fold it as it travels through the cartridge.

As seen in FIGS. 1, 2 and 3, prior art cartridge 10 includes a nozzle 18 terminating at a tip 20. It is the nozzle 18 that is inserted into the corneal incision and through which IOL 16 is ultimately expressed. As seen in FIG. 3, cartridge 10 has an interior bore 22 communicating with a nozzle passage 24 along which IOL 16 is passed. The '283 patent includes a full description of the shaping of bore 22 which accomplishes the folding of IOL 16 as it passes along bore 22 and ultimately, through nozzle 18 and tip 20 via nozzle passage 24.

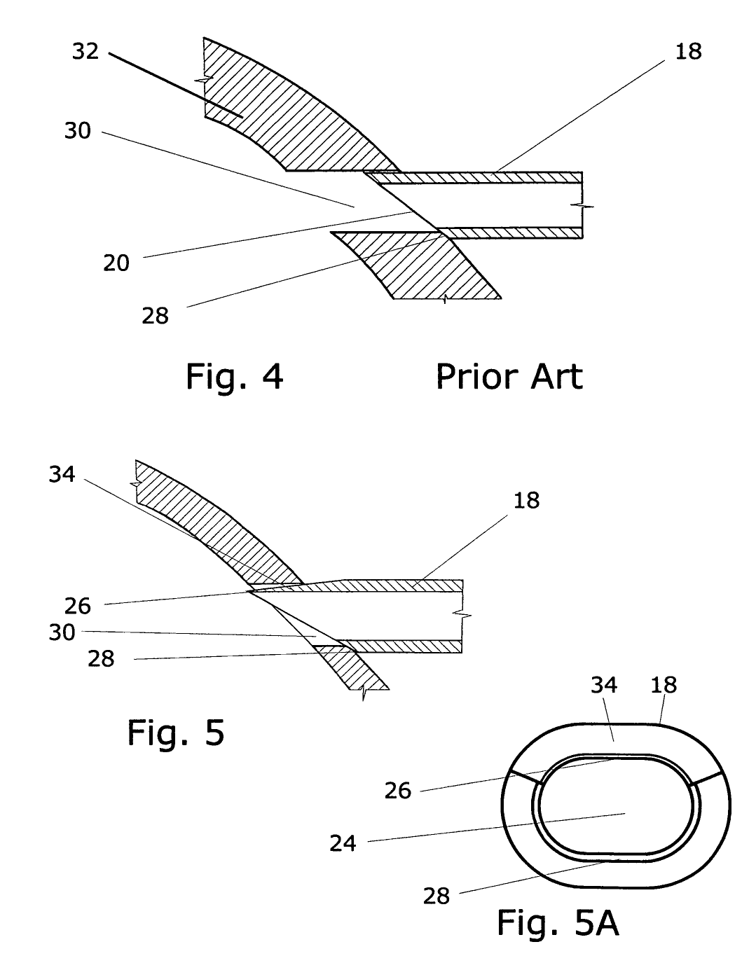

As seen in FIG. 1, tip 20 is formed by cutting or otherwise shaping nozzle 18 at an angle, resulting in a lead edge 26 and a trailing edge 28. For the purposes of the examples presented herein, the lead edge is that part of the rim forming the nozzle opening that is the farthest in an axial direction from body 12, while the trailing edge is that portion of the rim that is the closest in an axial direction to body 12. As seen schematically in FIG. 4, when nozzle 18 is positioned at incision 30, formed through cornea 32, lead tip edge 26 is typically inserted into incision 30 and thereafter, trailing tip edge 28 contacts incision 30.

As illustrated in FIG. 4, where incision 30 is slightly smaller than the outside diameter of nozzle 18, the surgeon may encounter some difficulty in passing nozzle 18 through incision 30, due to the contact between tip 20 and cornea 32. FIG. 4 illustrates the situation that can occur when trailing edge 28 contacts the exterior surface of cornea 32.

Referring to FIG. 5, a first embodiment of the present invention provides a taper 34 to an exterior portion of nozzle 18. Although the prior art cartridge shown in FIGS. 1, 2, 3 and 4 is illustrated as a specific device in the '283 patent, it is to be understood that the configurations shown as preferred embodiments in the present description applies to this tip and others as well.

In the preferred embodiment shown in FIG. 5, tapered nozzle section 34 extends about a portion of the circumference of nozzle 18, preferably including that portion of nozzle 18 which forms leading edge 26. As seen in FIG. 5 when leading edge 26 is inserted into incision 30, tapered nozzle section 34 allows nozzle 18 to be inserted into incision 30 even though incision 30 may close to slightly smaller in size than nozzle 18. As tapered nozzle section 34 enters incision 30 it exerts less force than if the taper were not present.

As seen in FIG. 5A, tapered section 34 covers a selected portion of the upper exterior surface of nozzle 18 with the remaining surface being without a taper. As seen in FIG. 5A tapered section 34 preferably includes lead edge 26.

In another embodiment of the present invention, the length of tapered nozzle section 34 may be selected to enable that portion of nozzle 18 to pass through incision 30 without reaching the full diameter of nozzle 18. As an example, a typical corneal incision tunnel is about 1 mm in length. Making tapered nozzle section 34 about 2 mm in length will allow section 34 to pass through incision 30 without being stretched to the full diameter of nozzle 18.

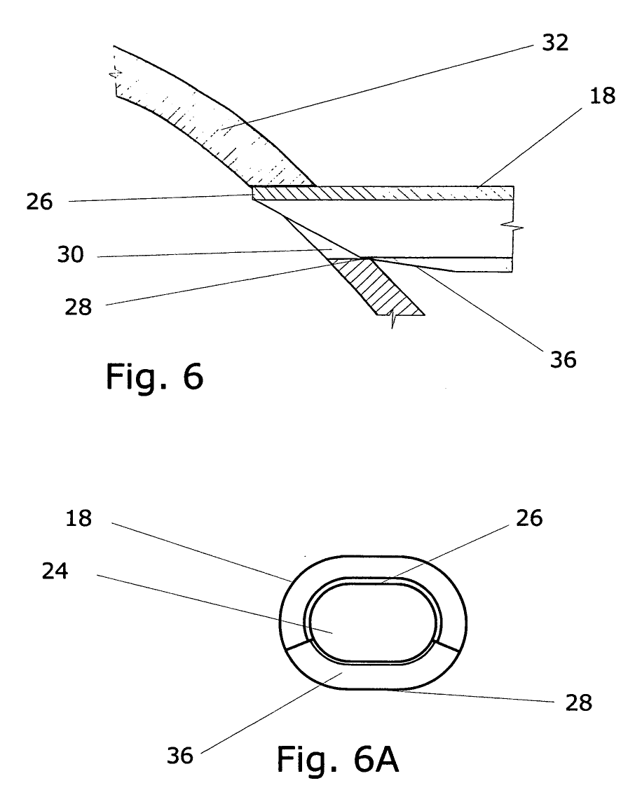

Referring now to FIG. 6, a second preferred embodiment of the invention is shown wherein a tapered section 36 is formed on the lowermost portion of nozzle 18, preferably including trailing edge 28. With this type of configuration an incision 30 large enough to accommodate lead edge 26 yet closed to a size less than the full diameter of nozzle 18 can be entered and opened gradually by tapered section 36.

As seen in FIG. 6A tapered section 36 covers a selected portion of the lower external surface of nozzle 18 and preferably includes trailing edge 28. The remaining portion of nozzle 18 remains untapered.

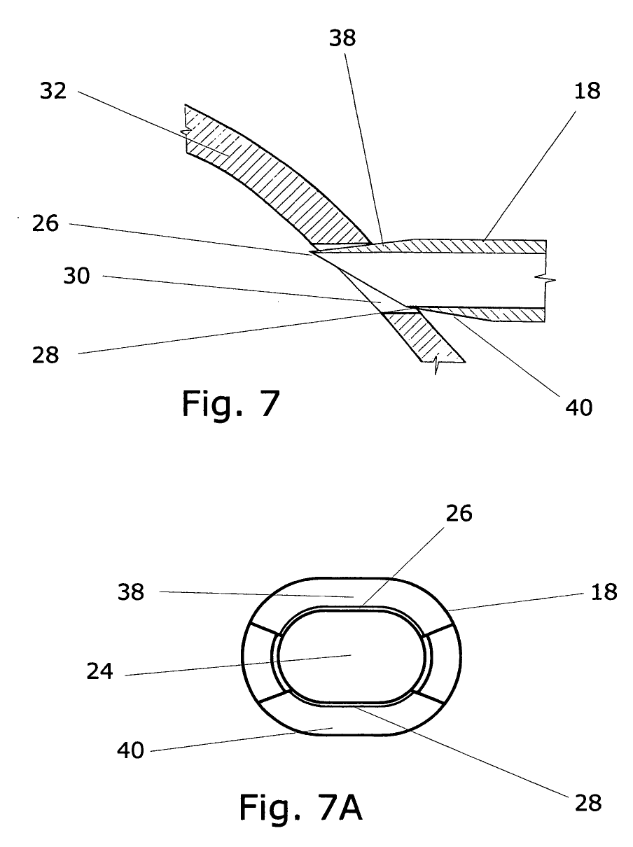

Referring now to FIG. 7 a third preferred embodiment of the present invention is shown wherein nozzle 18 has formed thereon an upper tapered section 38 and a lower tapered section 40, thought to facilitate entry into particularly tight incisions 30. When combining such an upper and lower taper consideration must be given to maintaining the rigidity of nozzle 18 by not removing too much material to form tapers 38, 40. It is contemplated that with a sufficiently rigid material a taper can be formed about the entire periphery of nozzle 18.

Referring to FIG. 7A upper tapered section 38 is seen to include lead edge 26, while lower tapered section 40 is seen to include trailing edge 40.

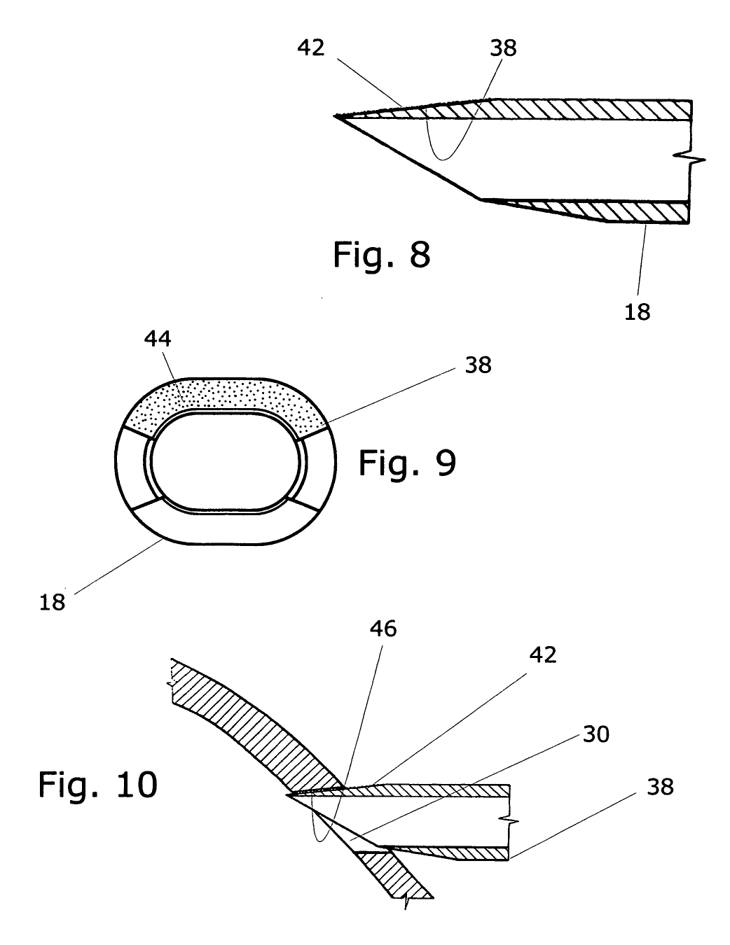

Referring now to FIG. 8, the numeral 42 identifies a textured surface formed on taper 38 of nozzle 18. Textured surface 42 is preferably formed by roughening the surface, such as by sandblasting but, as seen in FIG. 9, textured surface 42 can also be molded as a series of “bumps” 44 extending above the surface of taper 38. Other shapes, such as raised ridges can also be used to create the desired texturing.

Referring now to FIG. 10 nozzle 38 is shown extended into incision 30 with textured surface 42 in contact with the upper edge 46 of incision 30. Textured surface 42 provides a sufficiently non-slip surface to decrease the possibility that nozzle 18 will slip when inserted into incision 30.

While the foregoing describes a preferred embodiment or embodiments of the present invention, it is to be understood that this description is made by way of example only and is not intended to limit the scope of the present invention. It is expected that alterations and further modifications, as well as other and further applications of the principles of the present invention will occur to others skilled in the art to which the invention relates and, while differing from the foregoing, remain within the spirit and scope of the invention as herein described and claimed. Where means-plus-function clauses are used in the claims such language is intended to cover the structures described herein as performing the recited functions and not only structural equivalents but equivalent structures as well. For the purposes of the present disclosure, two structures that perform the same function within an environment described above may be equivalent structures.

I claim:

1. In a cartridge for holding a foldable intraocular lens, said cartridge of the type having a cartridge body having a cavity within which said lens is held, said cartridge body having a hollow nozzle communicating with said cartridge body cavity through which said lens is passed from said cartridge body cavity to exit said cartridge, said cartridge body cavity and said hollow nozzle aligned along a cartridge axis, the improvement comprising:
said nozzle having an external surface,
said nozzle having a nozzle tip formed proximate said nozzle opening,
said nozzle tip having at least one tapered surface extending about at least one portion of said external surface and tapering outwardly from said nozzle opening along said external surface.

2. The apparatus as recited in claim 1 wherein said nozzle tip is angled to form a leading edge and a following edge,
said leading edge being axially relatively further from said cartridge body than said following edge.

3. The apparatus as recited in claim 2 wherein said tapered surface includes and extends from said leading edge.

4. The apparatus as recited in claim 3 wherein said tapered surface includes and extends from said trailing edge.

5. The apparatus as recited in claim 3 wherein said nozzle has first and second tapered surfaces,
said first tapered surface including and extending from said leading edge; and
said second tapered surface including and extending from said trailing edge.

6. The apparatus as recited in claim 3 wherein said tapered surface is textured.

7. The apparatus as recited in claim 6 wherein said textured surface is formed by sandblasting.

8. The apparatus as recited in claim 6 wherein said textured surface is formed by molding raised bumps or ridges on said tapered surface.

9. The apparatus as recited in claim 5 wherein said first tapered surface is textured.

10. The apparatus as recited in claim 9 wherein said textured surface is formed by sandblasting.

11. The apparatus as recited in claim 9 wherein said textured surface is formed by molding raised bumps or ridges on said tapered surface.

12. In a cartridge for holding a foldable intraocular lens, said cartridge of the type having a cartridge body having a cavity within which said lens is held, said cartridge body having a hollow nozzle communicating with said cartridge body cavity through which said lens is passed from said cartridge body cavity to exit said cartridge, said cartridge body cavity and said hollow nozzle aligned along a cartridge axis, the improvement comprising:
said nozzle having an external surface,
said nozzle having a nozzle tip formed proximate said nozzle opening,
said nozzle having first and second tapered surfaces,
said first tapered surface including and extending from said leading edge; and
said second tapered surface including and extending from said trailing edge; and
said nozzle tip having at least one tapered surface extending about at least one portion of said first tapered surface and tapering outwardly from said nozzle opening along said external surface.

13. The apparatus as recited in claim 12 wherein said tapered surface is textured.

14. The apparatus as recited in claim 13 wherein said textured surface is formed by sandblasting.

15. The apparatus as recited in claim 13 wherein said textured surface is formed by molding raised bumps or ridges on said tapered surface
